(19) United States
(12) Patent Application Publication
(10) Pub. No.: US 2004/0220267 A1
(43) Pub. Date: Nov. 4, 2004

(54) Derivatives of pentacyclic nortriterpene quinone methides as compounds useful in the treatment of inflammatory, neurodegenerative, and neoplastic diseases

(76) Inventor: J. P. Devlin, Bridgewater, CT (US)

Correspondence Address: J. P. Devlin, Gaia Chemical, 23 George Washington Plaza, Gaylordsville, CT, 06755, US

(21) Appl. No.: 10/773,903

(22) Filed: Feb. 6, 2004

Related U.S. Application Data
(60) Provisional application No. 60/445,717, filed on Feb. 7, 2003.

(51) Int. Cl. A61K 31/203

(52) U.S. Cl. 514/559

(57) ABSTRACT

The uses of celastrol and pristimerin derivatives in the treatment of inflammatory, neurodegenerative and neoplastic diseases are disclosed, including dihydro derivatives of celastrol and pristimerin, such as dihydrocelastrol and dihydropristimerin and their diacetates.

### CROSS-REFERENCE TO RELATED APPLICATIONS

This application claims the benefit of U.S. Provisional Application No. 60/445,717, filed Feb. 7, 2003.

### FIELD OF THE INVENTION

The present invention relates to derivatives of celastrol and pristimerin, and the discovery that these compounds enhance the cellular production of heat shock proteins (molecular chaperones), and possess activities in the suppression of pro-inflammatory and inflammatory processes. The invention also relates to the use of these compounds as therapeutically effective agents in the treatment of inflammatory, neurodegenerative, and neoplastic diseases.

### BACKGROUND OF THE INVENTION

Numerous molecular and cellular events are associated with inflammatory disease processes. Such events may be defensive in the control or suppression of such processes, or may enhance inflammation to the detriment of the organism. The promotion of defensive mechanisms and the suppression of pro-inflammatory stimuli is the basis of therapy in the treatment of inflammatory, neurodegenerative, and neoplastic diseases.

All organisms constitutively express heat shock proteins (HSPs). HSPs (e.g, Hsp90, Hsp70, Hsp60, Hsp40 and Hsp28) which have basic and indispensable functions in the cell. Under stress conditions, such as inflammation and ischemia, HSP synthesis is elevated. These HSPs serve as molecular chaperones by assisting in the correct folding of stress-accumulated misfolded proteins, and preventing their aggregation and subsequent cellular damage. [S Leppa and L Sistonen, Ann Med 29: 73 (1997)]. HSPs are able to inhibit the aggregation of partially denatured proteins and refold them. Notably, Hsp70 has been shown to protect the brain and heart from severe ischemia. Therapeutic potential lies in the use of HSPs for protection against and therapeutic treatment of diseases caused by protein misfolding, such as neurodegenerative diseases. [K Ohtsuka and T Suzuki, Brain Res Bull 53: 141 (2000)]. Celastrol enhances cellular HSP production at sub-micromolar concentrations [RI Morimoto et al., (2004)].

Apoptosis (programmed cell death) is a normal aspect of tissue development, homeostasis and aging [A Glucksmann, Biol Rev Cambridge Philos Soc 26: 59 (1951)]. HSPs function at key regulatory points in the control of apoptosis [C Jolly and R I Morimoto, J Natl Cancer Inst 92:1564 (2000); V L Gabai and M Y Sherman, J Appl Physiol 92:1743 (2002)]. The molecular relationships between HSPs, various signaling proteins and partner proteins, regulate proliferation and development by preventing or enhancing cell growth and cell death as the levels of HSPs vary in response to stress or disease. [E A Nollen and R I Morimoto, J Cell Sci 115: 2809 (2002)]. HSPs are appropriate targets for modulating cell death pathways. [A Parcellier, S Gurbuxani, E Schmitt, E Solary and C Garrido, Biochem Biophys Res Commun 304:505 (2003)]. Celastrol has been reported to induce apoptosis in a human leukemia HL-60 model and inhibit topoisomerase II at sub-micromolar concentrations. [M Nagase, J Oto, S Sugiyama, K Yube, Y Takaishi and N Sakato, Biosci Biotechnol Biochem (Japan) 67: 1883 (2003)].

The pathogenesis of Alzheimer's disease is associated with: activation of microglia and the production of TNF-α, IL-1β, and superoxide. Also expressed is an inducible isoform of NO synthase which releases high levels of NO. In the presence of superoxide, NO forms peroxynitrite which damages neurons and other cell types. Celastrol inhibits microglial activation, the production of TNF-α, IL-1β, and the expression of inducible NO synthase. Celastrol does not inhibit constitutive NO synthases which is required to maintain neuronal function and vascular perfusion. [A C Allison, R Cacabelos, V R M Lombardi, X A Alvarez and C Vigo, CNS Drug Reviews 6: 45 (2000)]. Celastrol has also been shown to be a potent inhibitor of both NF-κB activation and nitric oxide production at IC₅₀'s of 0.27 and 0.23 uM respectively. This activity is considerably more potent than that of the standard, aminoguanidine with an IC₅₀ of 16 uM. [H Z Jin, B Y Hwang, H S Kim, J H Lee, Y H Kim and J J Lee, J Nat Prod 65: 89(2002)].

The accumulation of highly insoluble intracellular protein aggregates in neuronal inclusions is a hallmark of Huntington's and Parkinson's diseases as well as several other late-onset neurodegenerative disorders. Abnormal protein folding and aggregation play a key role in the pathogenesis of both HD and PD. Therefore, control of the molecular mechanisms of protein aggregation and its effects on neuronal cell death can provide new opportunities for therapy. [E E Wanker, Mol Med Today 2000: 387]. Celastrol is a potent inhibitor in the in vitro Huntington Aggregation Assay.

### SUMMARY OF THE INVENTION

Celastrol and pristimerin are related as acid and ester, respectively, on the same pentacyclic triterpene framework. Both compounds have shown serious cellular toxicity which has stifled development as useful drugs. With the belief that such toxicity can be decreased and useful activities enhanced by structural modifications, we have explored such modifications:

to define the structural features that independently govern potency and toxicity in one or more bioassays; and

to apply such information in the design of compounds with potential for therapeutic utility in inflammation and related diseases.

The above goals have been achieved by:

conducting a comparative evaluation of celastrol, pristimerin and known derivatives in relevant enzymatic and cellular systems; and

the design and synthesis of compounds related to celastrol and pristimerin that incorporate structural variations anticipated to provide a preferred biological activity profile.

This application addresses the structures of chemical derivatives of celastrol and pristimerin that, by virtue of the above analyses, leads to useful therapeutic candidates.

### THE INVENTION

Celastrol and pristimerin are both classified as pentacyclic nortriterpenes. Both compounds also possess a hydroxy quinone methide moiety extended by one additional double bond (1). It is this novel cluster of chemical functionality that is likely associated with biological activity.

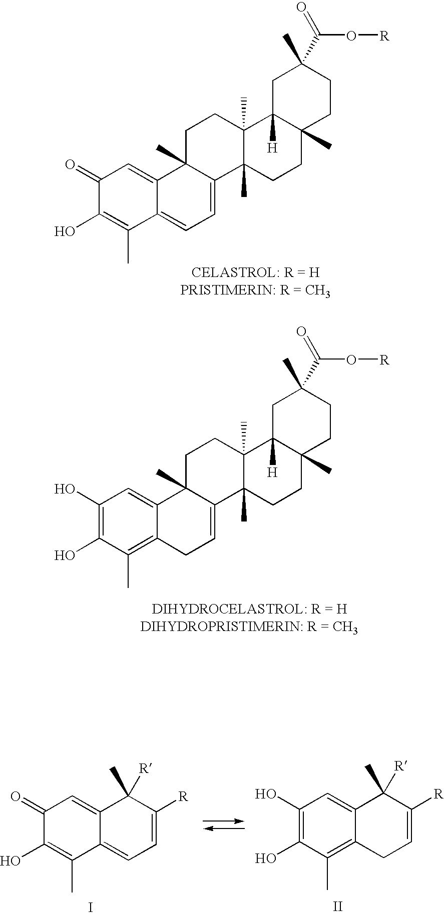

The literature (q.v.) describes several chemical modifications of celastrol and pristimerin. A facile conversion is the preparation of the dihydro derivatives, dihydrocelastrol and dihydropristimerin, respectively. Both of these derivatives are readily converted back to their parents by aerial oxidation. This facile and reversible conversion encouraged the exploration of dihydro derivatives as useful analogs that may bypass processes that impart untoward effects of these substances, and provide a means whereby they can regenerate the parent compounds at the cellular active site. There is little information available in the literature that relates to the comparative activities of derivatives of celastrol or pristimerin. One notable publication [M Nagase, J Oto, S Sugiyama, K Yube, Y Takaishi and N Sakato, Biosci Biotechnol Biochem (Japan) 67: 1883 (2003)] describes the ability of celastrol and dihydrocelastrol to induce apoptosis in a human leukemia HL-60 model, and also to inhibit topoisomerase II. In that report, dihydrocelastrol shows weaker activity than celastrol or the reference standard, etoposide. The rank order of potency was found to be celastrol>etoposide>dihydrocelastrol>> vehicle control.

Celastrol was isolated from the plant Celastrus scandens (Celastraceae) and recrystallized from hexane/toluene to yield red cubic crystals, mp 200-204° C. (lit: 204-205° C.) [K Nakanishi, Y Takahashi and H Budzikiewicz, J Org Chem 30: 1729 (1965)] Treatment of celastrol with an excess of ethereal diazomethane yielded, as previously described [K Nakanishi, H Kakisawa and Y Hirata, J Am Chem Soc 77: 3169, 6729 (1955)], the methyl ester, pristimerin, as orange needles, mp 215-219° C. [lit mp 219-220° C.].

Both celastrol and pristimerin are easily reduced with sodium borohydride according to published procedures [K Nakanishi, Y Takahashi and H Budzikiewicz, J Org Chem 30: 1729 (1965)] to yield the corresponding dihydro derivatives, dihydrocelastrol and dihydropristimerin.

Dihydrocelastrol diacetate: Acetylation of dihydrocelastrol with acetic anhydride/pyridine, and recrystallization of the product from toluene/hexane yielded dihydrocelastrol diacetate as pale yellow crystals, mp 224-226° C. (lit mp 211-212° C.). ¹H NMR (CDCl₃) 8 Me's, 1 double bond proton (5.7 ppm), 1 aromatic proton (6.9 ppm), 17 aliphatic protons, one exchangeable proton; 13C NMR (CDCl₃) 3 O═C═O, 8 aromatic and double bond signals, 22 aliphatic signals; MS: M−H @ 535, M+Cl @ 581, and M+Na @ 559. Purity (>95%) was confirmed by HPLC (UV & light scattering detectors).

Acetylation of dihydropristimerin with acetic anhydride/pyridine similarly yielded dihydropristimerin diacetate [mp 156-160° C.; lit¹ mp 164-165° C.].

2,3,29-triacetoxy-24-nor-friedela-1,3,5,7-tetraene [mp 103-106° C.] was prepared by LiAIH₄ reduction of pristimerin followed by acetylation as previously described [A W Johnson, P F Juby, T J King and S W Tam, J Chem Soc 1963: 2884].

Dihydro derivatives of celastrol and pristimerin have been shown to significantly enhance the cellular production of molecular chaperones, specifically Hsp70. Test data are presented in Table 1.

ENHANCEMENT OF Hsp70 GENERATION

| Dose (uM) | 0 | 0.1 | 0.3 | 0.5 | 1.0 | 2.0 | 3.0 | 6 | 10 |
|---|---|---|---|---|---|---|---|---|---|
| celastrol | .042 | .043 | .046 | .039 | .423 | 15.0 | 4.54 | .240 | .034 |
| dihydrocelastrol | .034 | .051 | .036 | .033 | .052 | .049 | .117 | 3.37 | .044 |
| dihydrocelastrol diacetate | .034 | .041 | .039 | .041 | .071 | 12.3 | 2.61 | .040 | .096 |
| pristimerin | .031 | .30 | .056 | .331 | 2.84 | .094 | .035 | .033 | 1.25 |
| dihydropristimerin | .038 | .050 | .039 | .037 | .043 | .042 | .045 | .102 | .034 |
| dihydropristimerin diacetate | .046 | .038 | .048 | .042 | .049 | .047 | .049 | .246 | .273 |

Dihydrocelastrol diacetate, in particular, is at least equivalent to celastrol in potency with reduced cytotoxicity. Dihydrocelastrol, dihydropristimerol and dihydrocelastrol diacetate were also potent in the inhibition of protein aggregation in the in vitro Huntington Aggregation assay.

The invention encompasses compounds of the general formulae Ill. Such compounds are useful for the treatment of inflammation, neoplastic diseases and related disorders.

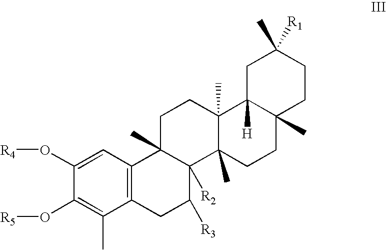

wherein R₁ can be H, CH₂OH, COOH, CH₂OCOR, wherein R is C-1 to C-12 alkyl, carboxyalkyl, carboxyalkenyl, alkoxycarbonylalkyl, alkoxycarbonylalkenyl, or aminoalkyl;

wherein R₂ and R₃ can individually be H or OH, or together as a double bond or epoxide; and wherein R₄ and R₅ can individually be H, lower acyl, or together can be a substituted or unsubstituted methylene or ethylene, —CO—, —COCO—, or —SO₂—.

What is claimed is:

1. In the treatment of inflammatory disease in a warm-blooded animal, the improvement which comprises administering a therapeutically effective amount of a compound of formula III to said animal.

2. In the treatment of neurodegenerative disease in a warm-blooded animal, the improvement which comprises administering a therapeutically effective amount of a compound of formula III to said animal.

3. In the treatment of neoplastic disease in a warm-blooded animal, the improvement which comprises administering a therapeutically effective amount of a compound of formula III to said animal.

4. The method as recited in claim 1 where in the compound of formula III R₁ is COOH, R₂ and R₃ are together as a double bond, and R4 and R5 are each Acetyl.

5. The method as recited in claim 2 where in the compound of formula III R₁ is COOH, R₂ and R₃ are together as a double bond, and R4 and R5 are each Acetyl.

6. The method as recited in claim 3 where in the compound of formula III R₁ is COOH, R₂ and R₃ are together as a double bond, and R4 and R5 are each Acetyl.
